(19) United States
(12) Patent Application Publication
(10) Pub. No.: US 2014/0073670 A1
(43) Pub. Date: Mar. 13, 2014

(54) PHARMACEUTICAL COMPOSITION COMPRISING FEXOFENADINE

(71) Applicant: AVENTIS PHARMACEUTICALS INC., BRIDGEWATER, NJ (US)

(72) Inventors: Sudhakara Rao BADABHAGNI, Guntur (IN); Nilesh JAISWAL, Bhopal (IN); Praveen KHULLAR, Dona Paula (IN); Kum PRASAD, Chanda Nagar (IN)

(73) Assignee: AVENTIS PHARMACEUTICALS INC., BRIDGEWATER, NJ (US)

(21) Appl. No.: 14/082,415

(22) Filed: Nov. 18, 2013

Related U.S. Application Data
(63) Continuation of application No. PCT/EP2011/059147, filed on May 16, 2012.
(60) Provisional application No. 61/499,856, filed on Jun. 22, 2011.

(30) Foreign Application Priority Data
May 20, 2011 (IN) 1727CHE2011
Jul. 13, 2011 (EP) 11305923.2

(51) Int. Cl. A61K 31/445

(52) U.S. Cl. CPC A61K 31/445
USPC 514/315

(57) ABSTRACT

The present invention relates to a pharmaceutical formulation of fexofenadine hydrochloride in a solvent system suitable as a liquid fill composition.

In another aspect, the invention also relates to a process for the preparation of the pharmaceutical formulation and the use of the composition for the treatment of allergic reactions in a patient.

### FIELD OF THE INVENTION

The present invention relates to a stable pharmaceutical composition of fexofenadine hydrochloride (HCl) for oral administration.

In particular, the invention pertains to an improved formulation comprising fexofenadine hydrochloride and pharmaceutically acceptable excipients, optionally encapsulated in a soft gelatin capsule.

The present invention furthermore also relates to a process for the preparation of such pharmaceutical composition and the use of such pharmaceutical composition for preparing a drug product for treating allergic reactions.

### BACKGROUND OF THE INVENTION

Fexofenadine has poor solubility in aqueous solution, and presents difficult problems in formulating such compounds for effective administration to patients. A well-designed formulation should, at a minimum, be capable of presenting a therapeutically effective amount of the hydrophobic compound to the desired absorption site, in an absorbable form. Even this minimal functionality is difficult to achieve when delivery of the hydrophobic therapeutic agent requires interaction with aqueous physiological environments, such as gastric fluids and intestinal fluids. Furthermore, drug absorption in different individuals might differ significantly due to differences in gastrointestinal function and food intake. Therefore, it is rather difficult to determine and control the dosage.

Fexofenadine [(+)-4-[1-hydroxy-4-[4(hydroxydiphenyl-methyl)-1-piperidinyl-butyl]-α,α-dimethyl benzene acetic acid] is an active metabolite of the second generation histamine H1 receptor antagonist (antihistamine) terfenadine. Fexofenadine is unique in that it appears to be purely non-sedating, even at higher doses in in-vitro models. Efflux transporter P-glycoprotein has been reported to transport fexofenadine and it is considered to be an important determinant of fexofenadine pharmacokinetics. Since fexofenadine is the substrate of P-gp and several organic anion transporting polypeptide (OATP), food and co-administration of drugs will have significant effect on its oral bioavailability. Further another challenge in the formulation of fexofenadine in oral administrable forms is the low solubility of fexofenadine, especially in gastric conditions (solubility of 0.2 mg of fexofenadine HCl per ml of pH 1.2 aqueous buffer solution).

Yet, another difficulty in the formulation of fexofenadine in oral pharmaceutical compositions is its unpleasant, strong and bitter taste and after taste which has led to poor, or even non-compliance with the treatment and thus has a negative impact on the efficiency of treatment.

Given the above, improving the absorption of orally administered drugs is the crucial point in solving the problem of low bioavailability of poorly soluble drugs.

To date, many methods have been employed to improve the bioavailability of poorly soluble drugs, such as converting them into soluble salts or esters, reducing the particle size and increasing the surface area to enhance drug dissolution, addition of solubilizing agents and the like. Moreover, although the active ingredient can be converted to soluble salts for drug administration, said soluble salts may revert back to poorly soluble forms due to the pH change in gastrointestinal tract, thus resulting in precipitation of drugs.

Also, fexofenadine hydrochloride faces reduced oral bioavailability (up to 33%) due to first pass metabolism due to involvement of P-Glycoprotein metabolic pathway.

Hence, there exists a need to circumvent the aforementioned drawbacks of reduced bioavailability and concurrently increase the rate of absorption in order to accelerate the biological availability of the medicament to the maximum, to achieve a very rapid pharmacological action, while having a stable composition.

U.S. Pat. No. 4,929,605 describes a pharmaceutical composition for oral administration comprising piperidinoalkanol compound and a nonionic surfactant such as polysorbate 80 (Tween 80) for increasing absorption and bioavailability of piperidinoalkanol compound. This document does not describe or suggest a pharmaceutical composition comprising fexofenadine hydrochloride and a liquid mixture of at least one non-ionic hydrophilic surfactant and at least one non-ionic hydrophobic surfactant. Furthermore, this document fails to address the technical problem of improving the storage stability and the shelf-life of a pharmaceutical composition comprising piperidinoalkanol compound.

WO99/08690 discloses a method for enhancing the bioavailability of the fexofenadine hydrochloride by oral co-administration of a p-glycoprotein inhibitor such as polyethylene glycol (PEG 400 or PEG 1000) or polysorbate. There is no mention of any pharmaceutical composition comprising fexofenadine hydrochloride and a liquid mixture of at least one non-ionic hydrophilic surfactant and at least one non-ionic hydrophobic surfactant. Furthermore, it was observed by the inventors that a combination of fexofenadine hydrochloride with only a non-ionic hydrophilic surfactant is not stable over the time, exhibits a decomposition of fexofenadine hydrochloride and has a less bioavailability than a composition according to the invention.

It is thus an object of the present invention to provide an orally administrable liquid pharmaceutical composition that improves the solubility and bioavailability of fexofenadine hydrochloride and that is stable over the time.

Applicants have found that this object can be achieved by providing an improved liquid composition comprising fexofenadine hydrochloride and pharmaceutically acceptable excipients.

It has been found surprisingly that the solubility and the bioavailability of the fexofenadine hydrochloride composition according to the invention is substantially more compared to the already known liquid formulations.

It has further been found that a capsule form containing this formulation reduces the taste of the residual remnants of the medicament.

### SUMMARY OF THE INVENTION

The present invention provides an orally administrable stable liquid pharmaceutical composition, comprising fexofenadine hydrochloride by compositely establishing optimal conditions for enhancing bioavailability of the drug, such as the co-relation between the drug and the accompanied components, selection of optimal mixing ratio of the respective components and use of specific surfactants, water content and pH regulating agents.

The instant formulation comprises a liquid mixture of at least one hydrophilic surfactant and at least one hydrophobic surfactant and one or more pharmaceutically acceptable excipients to produce a palatable and stable formulation with rapid therapeutic action, better absorption and bioavailability.

It is another object of the present invention to provide preparations such as a soft capsule having the improved disintegration degree and dissolution rate improved while having the bioavailability increased. Particularly, the invention pertains to preparing a soft gelatin capsule formulation of fexofenadine hydrochloride which in fine allows the obtaining of pharmacokinetic parameters bioequivalent to those which are obtained with conventional oral solid formulations of fexofenadine hydrochloride, for example tablets such as those available under the trademark Allegra®.

It is yet another object of the present invention to provide a pharmaceutical formulation, for instance, a soft capsule, a hard capsule, two-piece capsule or tablet comprising the formulation of the instant invention with improved chemical stability.

Another object of the invention is to provide a method for preparing the oral pharmaceutical composition of the invention, comprising dissolving the fexofenadine hydrochloride in an appropriate amount of a liquid mixture of at least one hydrophilic surfactant and at least one hydrophobic surfactant and bringing the pH to an acceptable range whereby the storage stability and the shelf-life of the formulation are enhanced.

The invention also relates to the use of the oral pharmaceutical composition of the invention for the preparation of a drug for the treatment of allergic reactions in a patient.

These and other aspects, features and advantages of the present invention will become better understood with reference to the following description and claims.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedence over any dictionary or extrinsic definition. Unless otherwise required by context, singular terms shall include the plural and plural terms shall include the singular.

According to the invention, a stable formulation means a formulation which, in particular, exhibits high resistance against decomposition of fexofenadine hydrochloride. Thus, upon storage for 3 months at 40 deg. C. and 75% humidity, the pharmaceutical composition according to the present invention usually does not exhibit any sign of high level of decomposition (with a total impurity level less than 1% by weight of the fexofenadine hydrochloride) and contains at least 99% by weight of the initial fexofenadine hydrochloride content (as evidenced by HPLC analysis).

The present invention employs a solvent system which is accomplished by compositely considering various factors, including optimal conditions for enhancing bioavailability of the drug, such as the co-relation between the drug and the accompanied components, selection of optimal mixing ratio of the respective components and use of specific surfactants, water content and pH regulating agents.

The exact dose of active agent and the particular formulation to be administered depend on a number of factors, e.g., the condition to be treated, the desired duration of the treatment and the rate of release of the active agent. For instance, the amount of the fexofenadine hydrochloride required and the release rate thereof may be determined on the basis of known in vitro or in vivo techniques, determining how long a particular active agent concentration in the blood plasma remains at an acceptable level for a therapeutic effect.

Accordingly, the first aspect of the invention relates to a pharmaceutical composition comprising fexofenadine hydrochloride with pharmaceutically acceptable excipients that has substantially more bioavailability.

In the composition of the present invention, the fexofenadine hydrochloride is present in amounts ranging from 1% to 35% by weight of the composition. In a most preferred embodiment the fexofenadine hydrochloride is present in amounts ranging from 10% to 30% by weight of the composition.

In the composition of the present invention, the fexofenadine hydrochloride has a preferred specific surface area ranging from 1.0 and 4.0 m²/g. In a most preferred embodiment the fexofenadine hydrochloride has a specific surface area of 3.2 m²/g.

In the composition of the present invention, the fexofenadine hydrochloride has a preferred particle size distribution (by Malvern) of D(0.1) 0.913 μm (diameter where 90% of the distribution is above and 10% is below); D(0.5) 9.207 μm (the volume median diameter where 50% of the distribution is above and 50% is below) and D(0.9) 15.896 μm (the volume median diameter where 10% of the distribution is above and 90% is below).

The composition contemplates the employment of a liquid mixture of at least one non-ionic hydrophilic surfactant and at least one non-ionic hydrophobic surfactant that functions as an oily vehicle. The surfactant mixture is present in an amount sufficient to promote the beneficial effects contemplated by the present invention.

In one embodiment, the pharmaceutical composition is comprising a mixture of at least one non-ionic hydrophilic surfactant which corresponds to a surfactant having an hydrophilic lipophilic balance (HLB) value of from 10 to 18, preferably from 11 to 16; and at least one hydrophobic surfactant which corresponds to a surfactant having an HLB value of from 4 to 10, preferably from 4 to 6. The HLB system (Fiedler, H. B., Encyclopedia of Excipients, 5th ed., Aulendorf: ECV-Editio-Cantor-Verlag (2002)) attributes numeric values to surfactants, with lipophilic (or hydrophobic) substances receiving lower HLB values and hydrophilic substances receiving higher HLB values.

The total amount of non ionic surfactant is at least of 60%, and preferably from 65 to 90% by weight, based on the total weight of the composition. More preferably, the total amount of surfactant is from 65 to 85% by weight of the composition.

Preferred non-ionic hydrophobic surfactants employable in context of the present include but are not limited to propylene glycol laurate (Lauroglycol 90), propylene glycol monocaprylate (Capryol-90) and mixture thereof. The most preferred hydrophobic surfactant for including in the pharmaceutical composition is propylene glycol monolaurate (Lauroglycol 90) which has an HLB value of 4.

In the composition of the present invention, it is preferred that the hydrophobic surfactant is present at a level of at least of 30% by weight of the composition. According to an advantageous embodiment of the pharmaceutical composition of the invention, the hydrophobic surfactant is present in amounts ranging from 50% to 85% by weight of the composition. More preferably, the hydrophobic surfactant is present in amounts ranging from 60% to 85% by weight of the composition. In a most preferred embodiment, the hydrophobic surfactant is present in amounts ranging from 75% to 80% by weight of the composition.

Another preferred hydrophilic surfactant for including in the pharmaceutical composition is polysorbate 80 (polyoxyethylene sorbitan monooleate; Tween 80) which has an HLB value of 15.

In the composition of the present invention, it is preferred that the hydrophilic surfactant is present in amounts ranging from 1% to 40% by weight of the composition. Also preferred, the hydrophilic surfactant is present in amounts ranging from 1% to 15% by weight of the composition. In a most preferred embodiment, the hydrophilic surfactant is present in amounts ranging from 1% to 10% by weight of the composition.

In another preferred embodiment of the invention, the pharmaceutical composition is a mixture of at least propylene glycol laurate (Lauroglycol 90) (the non-ionic hydrophobic surfactant) and at least polysorbate 80 (the non-ionic hydrophilic surfactant).

In a further aspect, the present invention relates to an oral administrable formulation comprising fexofenadine hydrochloride and a liquid mixture of at least one hydrophilic surfactant and at least one hydrophobic surfactant wherein the weight ratio of fexofenadine hydrochloride to the liquid mixture of surfactant is from 1:1.5 to 1:8. In a preferred embodiment of the invention, the weight ratio of fexofenadine hydrochloride to the liquid mixture is from 1:2 to 1:7 and most preferably this ratio is equal to 1:4.

Another aspect of the present invention is the pH of the fexofenadine hydrochloride in a suitable pharmaceutical vehicle, in order to guarantee an appropriate storage stability of the pharmaceutical formulation and to improve its storage stability and its shelf-life. The best results have been achieved for pH values of between 4 and 9 and more preferably from 5 to 6.

According to the present invention, these pH values can be achieved by means of addition of expedient acidifying and basifying agents.

The basifying agent used in the present invention may be selected from calcium carbonate, magnesium hydroxide, gum acacia, dicalcium phosphate, potassium hydroxide, sodium acetate, potassium phosphate, sodium carbonate, triethanolamine, etc. and their combinations. In a preferred embodiment, the basifying agent is triethanolamine.

The acidifying agent used in the present invention may be selected from acetic acid, lactic acid, ascorbic acid, citric acid, phosphoric acid, oxalic acid, calcium chloride, ammonium hydroxide, etc. and their combinations.

The invention also relates to a method for preparing a pharmaceutical preparation comprising 1 to 35% (w/w) of fexofenadine hydrochloride and at least 60% (w/w) of a liquid mixture of at least one non-ionic hydrophilic surfactant and at least one non-ionic hydrophobic surfactant. This method comprises the following steps: dissolving the fexofenadine hydrochloride in a liquid mixture of at least one non-ionic hydrophilic surfactant and at least one non-ionic hydrophobic surfactant, with stirring, in order to obtain an homogeneous mixture; and then adjust the pH between 5-6 using sufficient quantity of an acidifying or a basifying agent.

One aspect of the invention provides for soft gelatin capsules which include a capsule shell comprising gelatin and/or plasticizers and, if desired or required, further auxiliary materials.

In developing the soft gelatin capsule for fexofenadine hydrochloride composition according to the present invention, it must be recognized that the capsule is a system comprised of the fexofenadine hydrochloride composition and the gelatin shell used to encapsulate the fexofenadine composition. As such, not only is the filled fexofenadine composition critical to produce the desired solubility and bioavailability but the gelatin shell formulation is also critical as it must be compatible with the fexofenadine hydrochloride composition. One skilled in the art would be aware of the potential fill-shell interactions which could result in both physical and chemical capsule instability. Accordingly, the gelatin shell formulation utilized to form the capsule for the fexofenadine composition is also preferred in the present invention.

In general, gelatin shell capsule formulation for soft gelatin capsules consist of raw gelatin and one or more ingredients which are added to plasticize the gelatin to produce a capsule to suitable hardness as required by design or by preference. Typical plasticizers include glycerin and sorbitol (example: Special MDF™ 85 from SPI Pharma). Also, sorbitan anhydrides and mannitol may also be utilized. Furthermore, other non-traditional ingredients may also be used to plasticize the gelatin.

The preferred gelatin formulation for use in constructing soft gelatin capsules for use with the fexofenadine composition of the present invention includes gelatin and a plasticizer. Such plasticizers, which are well known in the pharmaceutical formulation art, include, for example, propylene glycol, and sorbitol.

The capsule formulations can also include other suitable additives such as preservatives or coloring agents which are utilized to stabilize the capsule or impart a specific characteristic such as color or look to the capsule. Pharmaceutically acceptable preservatives can include, for example, methyl and propyl parabens. Color may be imparted to the gelatin shell using FD&C or D&C dyes. Exemplary dyes include but are not limited to Tartrazine yellow, Azura red and the like. Opacifiers, such as titanium dioxide or iron oxides, may be employed to color or render the capsule opaque.

The invention contemplates use of coating agents which may include both non-functional or enteric coating agents such as cellulose based polymers, film coating agents or other coating agents known to a person of skilled in the art.

Other additives conventionally used in pharmaceutical compositions can be included, and these additives are well known in the art. Such additives include antioxidants, preservatives, chelating agents, complexing agents, viscomodulators, tonicifiers, flavorants, colorants odorants, opacifiers, suspending agents, binders, and mixtures thereof. The amounts of such additives can be readily determined by one skilled in the art, according to the particular properties desired. The present invention also contemplates the use of other pharmaceutical excipients such as binders, disintegrants, diluents, lubricants, plasticizers, permeation enhancers and solubilizers known to a person skilled in the art.

The core ingredients of a typical formulation according to the present invention may comprise:
1 to 35% (w/w) of fexofenadine hydrochloride;
At least 60% (w/w) of a liquid mixture of at least one hydrophilic surfactant and at least one hydrophobic surfactant that functions as an oily vehicle.

The gelatin shell ingredients of a typical formulation according to the present invention may comprise:
From 35% to 50% (w/w), more preferably 40-44% of gelatin;
From 15% to 30% (w/w), more preferably 15-25% of sorbitol in combination with glycerin;
From 0.1% to 10% (w/w) of coloring agents;
From 0.1 to 10% (w/w) of tartaric acid;
From 10 to 50% (w/w) of purified water.

The pharmaceutical compositions of the present invention can be prepared by conventional methods well known to those skilled in the art. However, the specific method of preparation will depend upon the ultimate dosage form. The composition can prepared by simple mixing or stirrer of the components to form a pre-concentrate. The hydrophobic therapeutic agent can be present in a first amount solubilized by the carrier, and a second amount in the carrier, as desired. It should be emphasized that the order of addition of the various components is not generally important and may be changed as convenient. Thereafter, the pH is brought to an preferably acceptable range where the stability is more and the mixture is shaken until a transparent solution is achieved.

Soft gelatin capsules are manufactured using rotary die process utilizing gelatin in a conventional process. Dry gelatin granules are combined with water and suitable plasticizers and the combination is then mixed and heated under vacuum to form a molten gelatin mass. The gelatin mass is held in its molten stage while being formed or cast into films or ribbons on casting wheels or drums. The films or ribbons are fed under the wedge and between rotary encapsulation dies. Within the encapsulation dies, capsules are simultaneously formed in pockets in the dies from the films or ribbons. The composition containing fexofenadine is filled into the soft gelatin capsules using any conventional method. The capsule is then cut and sealed. The seals are formed via a combination of pressure and heat as the capsule is filled and cut.

In another aspect, the present invention relates to provide a method to increase the bioavailability of the fexofenadine hydrochloride, which comprises the steps of: a) providing a stable gelatin composition comprising the liquid composition of the invention for oral administration; and b) administering said composition to said host for ingestion, whereby said composition contacts the biological fluids of the body and increases the bioavailability of the pharmaceutical active agent in order obtain pharmacokinetic parameters bioequivalent to those which are obtained with conventional oral solid formulations of fexofenadine hydrochloride, for example fexofenadine hydrochloride tablets such as those available under the trademark Allegra®.

Preferably the fexofenadine hydrochloride release rate of the composition of the invention filled into a gelatin shells and subjected for dissolution, when tested in FeSSIF-dissolution media (pH 5.8) with pancreatin in 500 ml, at 75 RPM and at 37° C., is at least of 40% (w/w) of the fexofenadine hydrochloride dissolved in 10 minutes and more than 50% of the fexofenadine hydrochloride dissolved in 15 minutes.

In use, the methods and compositions of the present invention contemplates a number of important advantages, including:

Robustness and improved delivery at the targeted site: The compositions of the present invention are unexpectedly robust and the compositions of the present invention unexpectedly provide improved delivery of the therapeutic agent to the absorption site, by minimizing precipitation. This improved delivery is believed to result in better bioavailability of the therapeutic agent.

Versatility: Compositions of the present invention can be carefully tailored and scaled to the polarity and functionality of the therapeutic agents, without compromising the improved solubilization, delivery, and other advantages as described above.

Ease of Preparation: The methods of the present invention provide compositions in which the hydrophobic therapeutic agent is readily solubilized, thereby conserving expensive manufacturing and personnel resources.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### EXAMPLES

### Example 1

### Composition According to the Invention

| | Quantity | | Quantity | | Quantity | |
|---|---|---|---|---|---|---|
| | (mg)/ | % | (mg)/ | % | (mg)/ | % |
| Component | capsule | w/w | capsule | w/w | capsule | w/w |
| Fexofenadine | 30.0 | 20.0 | 60.0 | 20.0 | 180.0 | 20.0 |
| HCl * | | | | | | |
| Propylene | 115.5 | 77.0 | 231.0 | 77.0 | 693.0 | 77.0 |
| glycol | | | | | | |
| monolaurate | | | | | | |
| Polysorbate 80 | 4.5 | 3.0 | 9.0 | 3.0 | 27.0 | 3.0 |
| Triethanol- | q.s. | | q.s. | | q.s. | |
| amine | | | | | | |
| Total | 150.0 | | 300.0 | | 900.0 | |

Gelatin shell composition

| | |
|---|---|
| Gelatin | 42% w/w |
| Sorbitol | 24% w/w |
| Coloring | 1.0 w/w |
| agents | |
| Tartaric acid | 0.75 w/w |
| Purified water | 33% w/w |

* with a specific surface area of 3.2 m²/g

All the excipients were dispersed. Fexofenadine hydrochloride was dispersed along with polysorbate 80 in propylene glycol monolaurate (Lauroglycol) (under continuous stirring). The mixture was stirred for 45 minutes. The pH of the resultant mixture was adjusted to a pH of 5 to 6 with triethanolamine if required. The formulation was encapsulated in a soft gelatin capsule at the fill weight of 900 mg for 180 mg strength according to one of the methods known per se to those skilled in the art.

### Example 2

### Composition According to the Invention

| | Quantity | | Quantity | |
|---|---|---|---|---|
| Component | (mg)/capsule | % w/w | (mg)/capsule | % w/w |
| Fexofenadine HCl* | 30.0 | 12.0 | 60.00 | 12.0 |
| Propylene Glycol | 195 | | 415 | |
| Monocaprylate | | 78.0 | | 78.0 |
| Propylene Glycol | 25 | 10.0 | 25 | 10.0 |
| Total | 250 | | 500 | |

Gelatin shell composition

| | |
|---|---|
| Gelatin | 45% w/w |
| Sorbitol | 20% w/w |
| Coloring agents | 0.25% w/w |
| Tartaric acid | 0.75% w/w |
| Purified water | 34% w/w |

*with a specific surface area of 3.2 m²/g

All the excipients were dispersed. Fexofenadine hydrochloride was dispersed along with propylene glycol monocaprylate (Capryol-90) under continuous stirring with application of heat till 125° C.-165° C. till a clear solution is formed. The resultant mixture was cooled till room temperature. The formulation was encapsulated in a soft gelatin capsule according to one of the methods known per se to those skilled in the art.

### Example 3

### Stability Study of the Composition According to the Invention

The pharmaceutical composition for oral administration tested is based on the following formula (fill composition A):

| Ingredient | Function | Mg/capsule | % w/w |
|---|---|---|---|
| Fexofenadine | Active Ingredient | 180.00 | 30.0 |
| HCL* | | | |
| Lauroglycol-90 | Lipophilic surfactant | 384.00 | 64.0 |
| Tween 80 | Hydrophilic surfactant | 36.00 | 6.0 |

| | | |
|---|---|---|
| Triethanolamine | pH adjuster | Q.S to pH 5-6 |

| | | |
|---|---|---|
| Total weight | 600 | mg |

*with a specific surface area of 3.2 m²/g

### Manufacturing Process:

1. Mix fexofenadine, Lauroglycol 90, Tween 80 in stainless steel vessel for 15 minutes;
2. Adjust the pH between 5-6 using sufficient quantity of triethanolamine;
3. Fill a soft gel capsule with the mixture obtained in step 2 using one of the methods known per se to those skilled in the art;
4. Pack the final pharmaceutical form in Alu/Alu blisters or PVC/PVdC pack.

### Stability Data of Fexofenadine HCL Soft Gelatin Capsules 60 mg (Homothetic Formula Based on the Fill Composition A:

| | | | | | Highest | | |
|---|---|---|---|---|---|---|---|
| | | | | Unknown | unknown | Total | Assay |
| Conditions | Imp-A | Imp-B | Imp-C | Imp. 1 | ump. | Impurities | (%) |
| | Related Substances (% w/w) of Fexofenadine HCL soft | | | | | | |
| | gelatin capsules packed in Clear Triplex Alu Blister pack. | | | | | | |
| Initial | 0.016 | 0.007 | 0.014 | 0.009 | 0.009 | 0.046 | 99.1 |
| 1 M/40° C. &75% RH | ND | 0.015 | 0.016 | 0.017 | 0.017 | 0.061 | 100.0 |
| 2 M/40° C. &75% RH | 0.037 | 0.015 | 0.016 | 0.012 | 0.012 | 0.080 | 100.0 |
| 3 M/40° C. &75% RH | 0.083 | 0.121 | 0.129 | 0.078 | 0.129 | 0.894 | 100.2 |
| | Related Substances (% w/w) of Fexofenadine HCL soft | | | | | | |
| | gelatin capsules packed in Alu/Alu Blister. | | | | | | |
| 1 M/40° C. &75% RH | ND | 0.015 | 0.014 | 0.013 | 0.013 | 0.054 | 98.7 |
| 2 M/40° C. &75% RH | ND | 0.012 | 0.015 | 0.012 | 0.012 | 0.039 | 99.8 |
| 3 M/40° C. &75% RH | ND | 0.010 | 0.016 | 0.009 | 0.009 | 0.035 | 98.6 |

### Stability Data of Fexofenadine HCL Soft Gelatin Capsules 30 mg (Homothetic Formula Based on the Fill Composition A:

| | | | | | Highest | | |
|---|---|---|---|---|---|---|---|
| | | | | Unknown | unknown | Total | Assay |
| Conditions | Imp-A | Imp-B | Imp-C | Imp. 1 | ump. | Impurities | (%) |
| | Related Substances (% w/w) of Fexofenadine HCL soft | | | | | | |
| | gelatin capsules packed in Clear Triplex Alu Blister pack. | | | | | | |
| Initial | ND | 0.011 | 0.014 | 0.014 | 0.014 | 0.039 | 101.0 |
| 1 M/40° C. &75% RH | 0.011 | 0.011 | 0.018 | 0.009 | 0.009 | 0.038 | 101.2 |
| 2 M/40° C. &75% RH | 0.040 | 0.020 | 0.017 | 0.015 | 0.015 | 0.092 | 99.0 |
| 3 M/40° C. &75% RH | 0.068 | 0.014 | 0.014 | 0.013 | 0.013 | 0.123 | 101.2 |
| | Related Substances (% w/w) of Fexofenadine HCL soft | | | | | | |
| | gelatin capsules packed in Alu/Alu Blister. | | | | | | |
| 1 M/40° C. &75% RH | ND | 0.017 | 0.019 | 0.014 | 0.014 | 0.050 | 99.4 |
| 2 M/40° C. &75% RH | ND | 0.012 | 0.017 | 0.013 | 0.013 | 0.042 | 99.5 |
| 3 M/40° C. &75% RH | ND | 0.013 | 0.016 | 0.013 | 0.013 | 0.042 | 99.9 |

### Stability Data of Fexofenadine HCL Tablets (Marketed Tablets Allegra® 30 mg):

| | Related Substances (% w/w) of Fexofenadine HCL Tablets (Marketed | | | | | |
|---|---|---|---|---|---|---|
| | tablets Allegra ® 30 mg) packed in Clear Triplex Alu Blister pack. | | | | | |
| | | | Unknown | Highest | Total | Assay |
| Conditions | Imp-A | Imp-B | Imp. 1 | unknown ump. | Impurities | (%) |
| Initial | 0.1 | ND | ND | None | 0.2 | 101 |
| 1 M/40° C. &75% RH | 0.1 | ND | ND | None | 0.2 | 100.6 |
| 2 M/40° C. &75% RH | 0.1 | ND | ND | None | 0.2 | 101.3 |
| 3 M/40° C. &75% RH | 0.1 | ND | ND | None | 0.2 | 100.6 |

In the above tables, the impurity level is expressed by weight of the fexofenadine hydrochloride, and the assay correspond to the level of the drug expressed by weight of the initial fexofenadine hydrochloride content.

Upon storage for 3 months at 40° C. and 75% humidity, the pharmaceutical composition according to the present invention does not exhibit any sign of decomposition (low impurities level, i.e., less than or equal to 1%) and contains at least 99% of the initial fexofenadine hydrochloride content (as evidenced by HPLC analysis).

From the above observations the soft gelatin formulation is more stable than the marketed tablets of 30 mg as evident by the total amount of impurities after 3 month at 40° C. and 75% RH conditions (0.123% w/w of total impurities after 3M for soft gelatin capsules packed in Clear Triplex Alu Blister pack comparing to 0.2% w/w of total impurities for the Fexofenadine HCL Tablets packed in the same blister pack).

### Example 4

### Comparative In Vitro Dissolution Study Between the Fexofenadine Allegra® Tablet and a Composition According to the Invention or not

Different samples according to the invention were prepared for dissolution study and compared with drug release profile of the Allegra® marketed tablet.

### Fill Composition B, Composition According to the Invention:

| Ingredient | Function | Mg/capsule | % w/w |
|---|---|---|---|
| Fexofenadine HCL | Active Ingredient | 180.00 | 30.0 |
| Lauroglycol-90 | Hydrophobic surfactant | 360.00 | 60.0 |
| Tween 80 | Hydrophilic surfactant | 60.00 | 10.0 |

| | | |
|---|---|---|
| Triethanolamine | pH adjuster | Q.S to pH 5-6 |

| | | |
|---|---|---|
| Total weight | 600 | mg |

### Fill Composition C, Composition According to the Invention:

| Ingredient | Function | Mg/capsule | % w/w |
|---|---|---|---|
| Fexofenadine HCL | Active Ingredient | 180.00 | 30.0 |
| Lauroglycol-90 | Hydrophobic | 180.0 | 30.0 |
| | surfactant | | |
| Polyoxyl 40 Hydrogenated | Hydrophilic | 240.0 | 40.0 |
| Castor Oil (Cremophor ® | surfactant | | |
| RH 40, BASF) | | | |

| | | |
|---|---|---|
| Triethanolamine | pH adjuster | Q.S to pH 5-6 |

| | | |
|---|---|---|
| Total weight | 600 | mg |

### Fill Composition D, Comparative Example (Containing Only Non-Ionic Hydrophilic Surfactants and No Hydrophobic Surfactant):

| Ingredient | Function | Mg/capsule | | % w/w |
|---|---|---|---|---|
| Fexofenadine HCL | Active Ingredient | 180.0 | | 30.0 |
| PEG 400 | Hydrophilic surfactant | 180.0 | | 30.0 |
| Polyethoxylated castor | Hydrophilic surfactant | 198.0 | | 33.0 |
| oil (Cremophor ® | | | | |
| EL 30, BASF) | | | | |
| Other excipients | | 42.0 | | 7.0 |
| Total weight | | 600 | mg | |

### Manufacturing Process:

1. Mix Fexofenadine, hydrophilic surfactants and the other ingredients in stainless steel vessel for 15 minutes;
2. Adjust the pH between 5-6 using sufficient quantity of Triethanol amine;
3. Fill a soft gel capsule with the mixture obtained in step 2 in different gelatin composition using one of the methods known per se to those skilled in the art.

### Methodology:

### a) Instrumentation (or Equivalent)

An automated dissolution system comprising of a water bath to maintain the set temperature precisely and accurately, a group of dissolution bowls mounted on a plate in the water bath, a mechanism to stir the liquid content of the bowls and an automated sampling accessory to draw and replenish the liquid media into the vessel.

### b) Dissolution Test Apparatus:

(Manufacturer: LabIndia instruments Ltd., Model: Disso 2000)
A HPLC System comprising of a pump capable of delivering the mixture of solvents precisely and accurately attached to a column thermostat and a UV-Visible detector. The data generated should be captured by a software capable of processing the data from the above mentioned hardware.
Manufacturer: Agilent Technologies Ltd., Model: 1200 series with OpenLAB™ Disso 2000)

### c) Release Test Procedure

Equipment: 8-vessel assembly and paddles
Medium: As mentioned below.
Volume: 500 ml
Stirring Rate: 75 RPM
Temperature: 37° C.

### d) Procedure

Place one weighed capsule in each vessel containing required volume of medium maintained at 37° C. and immediately operate the apparatus for 60 minutes. Withdraw a fixed volume of samples at different time intervals and at the end of dissolution cycle. Filter each sample through 0.45 μm membrane filters. Replace an equal aliquot of media stored at 37° C.
Analyze the samples using HPLC by preparing Standard of equivalent concentration.

### e) Analysis Parameters on HPLC

Column: A column having a internal diameter of 4.6 mm and length 150 mm, packed with a hybrid silica particles having octyl chain attached to it. For example:
Waters C-8 150 mm×4.6 mm.
Column Temperature: Ambient
Mobile Phase: Phosphate Buffer (pH 3.0): Acetonitrile in the ratio of 60:40 V/V
Flow Rate: 1.5 ml/minutes
Detector: 220 nm (if UV, precise the wavelength)
Injection Volume: 10 μL

### f) Dissolution Media Composition: (Also Called as FeSSIF: Fed State Simulated Intestinal Media)

| Ingredients of Dissolution Media | Conc. |
|---|---|
| Sodium Taurocholate (mM) | 10 |
| Lecithin (mM) | 2 |
| Glyceryl monooleate (mM) | 5 |
| Sodium Oleate (mM) | 0.8 |
| Maleic Acid (mM) | 55.02 |
| Sodium Hydroxide (mM) | 81.65 |
| Sodium Chloride (mM) | 125.5 |
| pH | 5.8 |
| Osmolality (mOsm kg⁻¹) | 380-400 |

The above formulation fills B, C and D were filed into empty hard gelatin shells and subjected for Dissolution studies.

### Comparative In Vitro Dissolution Results:

| | Drug release of Fexofenadine HCL | | | | |
|---|---|---|---|---|---|
| | in FeSSIF-dissolution media (pH | | | | |
| | 5.8) (with Pancreatin)/ | | | | |
| | 75 rpm/Paddle/500 ml [% (w/w)] | | | | |
| Product tested | 10 min | 15 min | 30 min | 45 min | 60 min |
| Allegra ® Marketed | 77 | 84 | 92 | 95 | 98 |
| Tablets 180 mg | | | | | |
| Composition B | 63 | 66 | 70 | 73 | 79 |
| (invention) | | | | | |
| Composition C | 48 | 57 | 62 | 65 | 65 |
| (comparative example) | | | | | |
| Composition D | 16 | 27 | 48 | 60 | 67 |
| (comparative example) | | | | | |

The ultimate objective to formulate Fexofenadine HCL soft gel capsules was to make it bioequivalent to Allegra Tablets. For this as a pre-requisite it is very important first to match the dissolution profile of soft gelatin capsules Vs. Allegra tablets specially in pH 5.8 FeSSIF Media.

The above experimental fills which were formulated using different combinations of a non-ionic hydrophilic surfactant and with or without a non-ionic hydrophobic surfactant and were subjected to dissolution studies in biorelevant media reveal that there is an increase in release of the fexofenadine hydrochloride from the fill if the fill comprises a non-ionic hydrophilic surfactant combined with a non-ionic hydrophobic surfactant as evident with the fill composition B or C where 63% or 48% (w/w) of the fexofenadine hydrochloride dissolved is released after 10 min, respectively, comparing to a drug release of 16% (w/w) of the fexofenadine hydrochloride dissolved after 10 min for the fill composition D which do not contain hydrophobic surfactant.

Thus, with a fill composition according to the invention containing at least 60% (w/w) of a liquid mixture of at least one non-ionic hydrophilic surfactant and at least one non-ionic hydrophobic surfactant (fill composition B and C), the onset of action is more rapid by comparison to a composition which do not contain non-ionic hydrophobic surfactant (fill composition D).

The foregoing discussion and description are illustrative of some embodiments of the present invention, but are not meant to be limitations on the practice thereof.

What is claimed is:

1. A pharmaceutical composition for oral administration comprising:
1 to 35% (w/w) of fexofenadine hydrochloride; and
at least 60% (w/w) of a liquid mixture of at least one non-ionic hydrophilic surfactant and at least one non-ionic hydrophobic surfactant.

2. The pharmaceutical composition according to claim 1, wherein the fexofenadine hydrochloride has a specific surface area ranging from 1.0 and 4.0 m²/g.

3. The pharmaceutical composition according to claim 1, wherein the total amount of surfactant ranges from 65 to 85% by weight of the composition.

4. The pharmaceutical composition according to claim 1, wherein the non-ionic hydrophobic surfactant has an HLB value from 4 to 6.

5. The pharmaceutical composition according to claim 1, wherein the non-ionic hydrophobic surfactant is chosen from the group consisting of propylene glycol laurate, propylene glycol monocaprylate and mixtures thereof.

6. The pharmaceutical composition according to claim 1, wherein the hydrophobic surfactant is present in an amount ranging from 75% to 80% by weight of the composition.

7. The pharmaceutical composition according to claim 1, wherein the non-ionic hydrophilic surfactant has an HLB value from 11 to 16.

8. The pharmaceutical composition according to claim 1, wherein the non-ionic hydrophilic surfactant is polysorbate 80.

9. The pharmaceutical composition according to claim 1, wherein the non-ionic hydrophilic surfactant is present in an amount ranging from 1% to 10% by weight of the composition.

10. The pharmaceutical composition according to claim 1, wherein the weight ratio of fexofenadine hydrochloride to the total liquid mixture of surfactant is 1:4.

11. The pharmaceutical composition according to claim 1, wherein the non-ionic hydrophobic surfactant is propylene glycol monolaurate and the non-ionic hydrophilic surfactant is polysorbate 80.

12. The pharmaceutical composition according to claim 1, wherein the final pH value of the composition is between 4 and 9.

13. The pharmaceutical composition according to claim 1, in the form of a soft capsule.

14. A method for preparing a pharmaceutical preparation according to claim 1, comprising the following successive steps: dissolving the fexofenadine hydrochloride in a liquid mixture of at least one non-ionic hydrophilic surfactant and at least one non-ionic hydrophobic surfactant, with stirring, in order to obtain a homogeneous mixture; and then adjusting the pH to between 5-6 using a sufficient quantity of an acidifying or a basifying agent.

15. A method for the treatment of an allergic reaction in a patient comprising the administration of a pharmaceutically effective dose of a composition as claimed in claim 1 to the patient.
